(19) United States
(12) Patent Application Publication
(10) Pub. No.: US 2011/0306792 A1
(43) Pub. Date: Dec. 15, 2011

(54) PROCESS FOR PREPARING TOLTERODINE AND THE L-TARTRATE THEREOF

(75) Inventors: Gang Wang, Shanghai (CN); Jingzhong Zhang, Shanghai (CN); Xungui He, Shanghai (CN); Yuan Wang, Shangai (CN)

(73) Assignee: 2Y-CHEM, LTD., Shanghai (CN)

(21) Appl. No.: 13/138,061

(22) PCT Filed: Aug. 18, 2009
(86) PCT No.: PCT/CN2009/000950

§ 371 Date: Aug. 17, 2011

(30) Foreign Application Priority Data
Dec. 29, 2008 (CN) 200810208229.9

(51) Int. Cl. C07C 213/10

(52) U.S. Cl. 564/302

(57) ABSTRACT

The present invention relates to a process for preparing tolterodine and the L-tartrate thereof. The preparation consists of the following steps: A) ammonolysis reaction between diisopropylamine and compound 2 (3,4-dihydro-6-methyl-4-phenyl-2H-benzopyran-2-one) activated by an activator to afford the amide 3; B) reduction of the amide by a reductant to give compound 1, i.e., racemic tolterodine free base; C) Resolution of the tolterodine free base to afford tolterodine L-tartrate. The present route is very short and suitable for industrial production.

### TECHNICAL FIELD

The present invention relates to a process for preparing tolterodine [(R)-N,N-diisopropyl-3-(2-hydroxyl-5-methylphenyl)-3-phenylpropylamine] and the L-tartrate thereof.

### BACKGROUND OF THE INVENTION

Tolterodine (as shown in Formula 1) is the common name of the compound (R)-N,N-diisopropyl-3-(2-hydroxyl-5-methylphenyl)-3-phenylpropylamine, and the formula thereof is shown below:

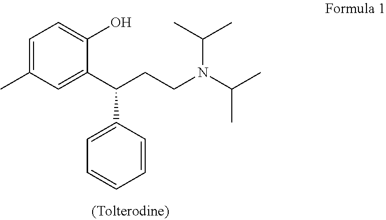

Tolterodine is developed by Pharmacia Co. Ltd., U.S., which is a competitive M (muscarinic) receptor antagonist with high selectivity to bladder. It can competitively combine with M receptor so as to block the combination of neurotransmitter acetylcholine and M receptor. Thus the contraction of detrusor muscle can be effectively inhibited and the symptoms such as pollakisuria, urgent micturition and pressing anischuria, etc. can be relieved accordingly. In addition, its metabolites exhibit higher selectivity to M receptor. Therefore, tolterodine has been widely used due to its good therapeutic effect and less side effects.

For example, a process for preparing tolterodine and its tartrate has been disclosed in U.S. Pat. No. 5,382,600, wherein the racemic tolterodine was achieved by a 6-step reaction: the cyclization between trans-cinnamic acid as the initial raw material and p-cresol to give 3,4-dihydro-6-methyl-4-phenyl-2H-benzopyran-2-one; then ring-opening by methylation with methyl iodide; reduction of ester group with lithium aluminum hydride; p-tosylation of hydroxyl; substitution with diisopropylamine and deprotection of methyl with boron tribromide. Finally, racemic tolterodine was resolved with L-(+)tartaric acid to give L-(+)tartrate of tolterodine.

In WO 98/29402, a preparation process using the same raw material as that used in the above US patent has been disclosed. In this process, racemic tolterodine free base was produced through 4 steps including: preparing 3,4-dihydro-6-methyl-4-phenyl-2H-benzopyran-2-one first; reducing it with diisobutyl aluminium hydride to obtain 6-methyl-4-phenylchroman-2-ol; performing the reduction and ammonolysis with diisopropylamine in the presence of Pt/C, then the racemic tolterodine free base was resolved with L-(+)tartaric acid to give the target product.

A synthesis process using 3,4-dihydro-6-methyl-4-phenyl-2H-benzopyran-2-one as the raw material has been disclosed in WO 2008/020332, wherein, racemic tolterodine was produced by 4 steps, i.e., the raw material was reduced with NaBH₄ to give 2-(3-hydroxy-1-phenylpropyl)-4-methylphenol, then it was mesylated, substituted with diisopropylamine etc., which was resolved to obtain the target product.

The reaction conditions of the above processes are strict, and the reactions are dangerous and difficult to operate, which makes the industrial production of tolterodine to be hardly realized.

A synthesis process using 3-bromo-N,N-diisopropyl-3-phenylpropanamide as the raw material has been disclosed in EP 1693361, wherein racemic tolterodine was prepared via 3 steps, i.e., 3-bromo-N,N-diisopropyl-3-phenylpropanamide was etherified with p-cresol in basic condition to give N,N-diisopropyl-3-phenyl-3-(4-tolyloxy)propanamide, then rearrangement and reduction were followed, after that, the racemic tolterodine was resolved to obtain the target product. Main drawback for this route is that the raw material is not readily available, that is, the synthesis of 3-bromo-N,N-diisopropyl-3-phenylpropanamide may involve four steps of reactions and the overall yield of this route is also low.

A synthesis process using cinnamaldehyde as the raw material has been disclosed in WO2007/138440, wherein, cinnamaldehyde reacted with p-cresol in the presence of N-methylpiperazine to give 6-methyl-4-phenylchroman-2-ol, then reductive amination and resolution were performed to obtain tolterodine tartrate. Although the route is simple, the chiral purity of tolterodine tartrate is only 91%.

Another synthesis process using cinnamyl chloride as the raw material has been disclosed in WO2007/147547, wherein the raw material was substituted with diisopropylamine to give N,N-diisopropylphenylpropenylamine which was then reacted with p-cresol using methanesulfonic acid as a catalyst to give racemic tolterodine, and the racemic tolterodine was resolved to obtain the target product. Cinnamyl chloride as raw material is expensive, thus the production cost increases, and etherified byproduct is easily generated during the amine-substituted step under the reacting condition, so the application has some limitations.

### SUMMARY OF THE INVENTION

In order to solve the above problems, the object of the present invention is to provide a process for preparing tolterodine [(R)-N,N-diisopropyl-3-(2-hydroxyl-5-methylphenyl)-3-phenylpropylamine] and the L-tartrate thereof. The process has simple procedure, low cost and high yield. In addition, it is easily operated and can provide stable product quality, so it is suitable for industrial preparation.

To attain the above object, the present invention is achieved by the following technical solution.

The process for preparing tolterodine and the L-tartrate thereof of the present invention comprises the following steps:

A) performing ammonolysis reaction between diisopropylamine and compound 2 (3,4-dihydro-6-methyl-4-phenyl-2H-benzopyran-2-one) after diisopropylamine was activated by an activator, to give compound 3 (N,N-diisopropyl-3-phenyl-3-(2-hydroxy-5-methylphenyl)propionamide); or performing ammonolysis reaction between diisopropylamine and 3,4-dihydro-6-methyl-4-phenyl-2H-benzopyran-2-one after they were activated by Lewis acid, to give N,N-diisopropyl-3-phenyl-3-(2-hydroxy-5-methylphenyl)propionamide;

and the corresponding reaction equation is shown as follows:

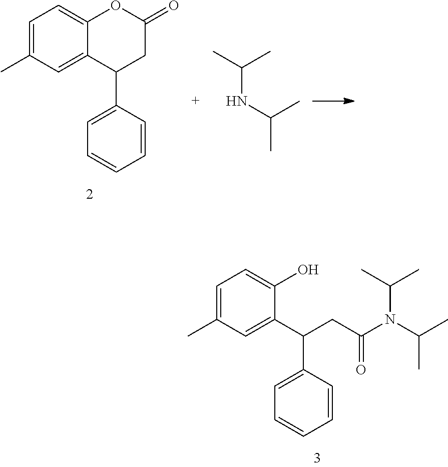

B) reducing compound 3 by a reductant to give compound 1, i.e., racemic tolterodine free base, and the corresponding reaction equation is shown as follows:

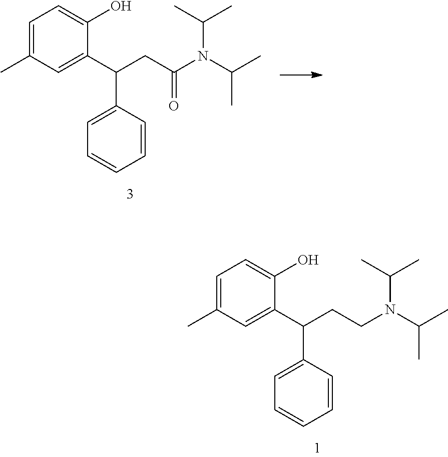

C) resolving compound 1 (racemic tolterodine free base) with L-(+) tartaric acid to give compound 4 (L-(+) tartrate of tolterodine), and the corresponding reaction equation is shown as follows:

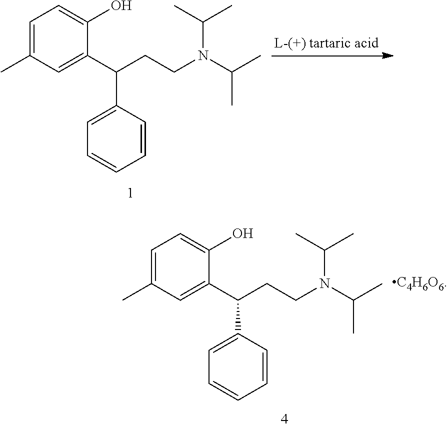

The process of the present invention will be described in more details as follows.

In the step A):

the activator for the activation of the diisopropylamine is an alkali metal reagent selected from lithium aluminium hydride, butyllithium and Grignard reagent; and the amount of the activator is regulated on the basis of the amount of the diisopropylamine accordingly, generally, the molar ratio of the activator to diisopropylamine is 1:2 to 1:1;

alternatively, Lewis acid, such as one of AlCl₃, ZnCl₂, BF₃, TiCl₄, Ti(O-i-Pr)₄ and SnCl₄ or the combination thereof, can be used to activate diisopropylamine and compound 2; and generally, the amount of the Lewis acid is that the molar ratio of the Lewis acid to diisopropylamine is 1:1.5 to 1:5; the reaction mechanism is that Lewis acid can combine with diisopropylamine to form a composite which can activate the lactone structure in compound 2, thus it is beneficial to the ammonolysis reaction of the remaining diisopropylamine and compound 2;

the solvent used in the reaction can be tetrahydrofuran, acetonitrile, dichloromethane, toluene, xylene, chloroform, dichloroethane or chlorobenzene etc.;

the proper temperature range of the reaction is from −80° C. to 130° C., and preferably from 10° C. to 70° C.;

generally, the molar ratio of compound 2 to diisopropylamine is 1:1 to 1:30, and preferably 1:2 to 1:10;

at the end of step A), in the case of using an activator to activate diisopropylamine, the reaction is quenched with a quenching reagent, then the reaction liquid is acidified until the pH value thereof reaches 2 to 4, and preferably 3, followed by extraction with an organic solvent, and then the resulting extracted organic phase is concentrated under reduced pressure, after that a crystallizing solvent is added therein for crystallization to obtain compound 3; while in the case of using Lewis acid to activate diisopropylamine and 3,4-dihydro-6-methyl-4-phenyl-2H-benzopyran-2-one, at the end of the reaction, it is quenched with water, then an organic phase is separated and concentrated under reduced pressure, after that a crystallizing solvent is added therein for crystallization to obtain compound 3; wherein:

the quenching reagent is anyone selected from water, aqueous solution of ammonia chloride, hydrochloric acid, sulfuric acid, formic acid, acetic acid, propionic acid, butyric acid, oxalic acid, citric acid and the combinations thereof; the acidification employs anyone of hydrochloric acid, sulfuric acid, formic acid, acetic acid, propionic acid, butyric acid, oxalic acid, citric acid and the combinations thereof; the organic solvent used for extraction is ethyl acetate, toluene, dichloromethane, methyl tert-butyl ether or the like; the crystallizing solvent may be anyone of ethyl acetate/petroleum ether, methanol, methanol/water, methyl tert-butyl ether/petroleum ether, methyl tert-butyl ether/heptane, dichloromethane/petroleum ether and the combinations thereof.

In the step B):

the reductant may be anyone of metal hydride and borohydride, or the combinations of said hydrides and Lewis acid; or anyone of Red-Al, diisobutyl aluminium hydride (DIBAL-H), BH₃ and its complexes, such as BH₃.(CH₃)₂S or BH₃.(CH₂)₄O, also can be used in step B) as the reductant;

alternatively, the reductant used in step B) may also be NaBH₄, KBH₄, or any combination between either of them and a Lewis acid selected from sulfuric acid, AlCl₃, ZnCl₂, BF₃.(CH₂)₄O and iodine (I₂);

at the end of the reduction reaction, it is quenched with water as the quenching reagent; then the reaction liquid is extracted with ethyl acetate or dichloromethane, the resulting organic phase is concentrated under reduced pressure to obtain racemic tolterodine free base.

In the preparation process of the present invention, compound 2, i.e., 3,4-dihydro-6-methyl-4-phenyl-2H-benzopyran-2-one, can be prepared according to the literature (Journal of the South African Chemical Institute, 1949, (2), 165-71).

### ADVANTAGEOUS EFFECTS OF THE INVENTION

The preparation process of the present invention has only three steps in total. The synthetic route is simple, and the raw material is intermediate 3,4-dihydro-6-methyl-4-phenyl-2,4-benzopyran-2-one, which is inexpensive and easy to get. In the ammonolysis reaction of the step A), the lactone structure is directly converted into amide in one step by effectively increasing the activity of the reaction substrate using an activator. The following reduction reaction and chiral resolution are reliable since there are many similar literatures. Each reaction in this synthetic route is regular one, therefore the operation and aftertreatment are easy, and so it is suitable for industrial production. The quality of final product is stable and measures up to the product quality requirement defined by the United States Pharmacopoeia.

### BEST MODE FOR CARRYING OUT THE INVENTION

The technical solution, feature and advantage of the invention are further illustrated below in examples, but the scope of the invention should not be limited by the examples.

### 1. Synthesis of Compound 3 (N,N-diisopropyl-3-phenyl-3-(2-hydroxy-5-methylphenyl)propionamide)

### Example 1

Mg powder (1.0 g, 0.042 mol) was heated to 65° C. in a 100 ml 3-neck flask under the protection of nitrogen, and 30 ml of tetrahydrofuran was added thereto after half an hour, then bromoethane (4.6 g, 0.042 mol) diluted by 10 ml tetrahydrofuran (wherein the reaction is initiated by 2 drops of bromoethane) was slowly added to the reaction flask dropwise and the reflux temperature was maintained at 65° C. After the Mg powder disappeared completely, diisopropylamine (6.4 g, 0.063 mol) was added thereto under reflux for 1 hour. Then the solution of compound 2 in tetrahydrofuran (5 g, 0.021 mol, in 10 ml THF) was added thereto under reflux overnight and the reflux temperature was maintained at 65° C. After the reaction was completed, it was quenched with 10 ml water. The pH value of the reaction solution was adjusted to 3 with concentrated HCl, then an organic phase was separated by adding 50 ml ethyl acetate, washed with water (30 ml×3) and dried. After that, the organic phase was concentrated under reduced pressure to remove solvent, then methanol with 10 times volume of the residue was added thereto for crystallization to obtain a solid subject product.

MS (ESI, m/z): [M+]⁺ 340.3.

¹H-NMR (400 MHz, CDCl₃): δ 7.31 (m, 5H); 6.84 (s, 2H); 6.63 (s, 1H, J=7.6 Hz); 4.99 (t, 1H), 4.05 (m, 1H); 3.45 (s, 1H); 3.11 (d, 2H); 2.11 (s, 3H); 1.07-1.37 (m, 12H).

### Example 2

Diisopropylamine (0.85 g, 0.0084 mol) was dissolved in 10 ml tetrahydrofuran in a 50 ml 3-neck flask under the protection of nitrogen. After the reaction mixture was cooled to −80° C., butyl lithium (3.36 ml, 0.0084 mol) was added thereto, and the temperature was maintained lower than −60° C., kept stirring for 30 minutes to obtain a solution of LDA (lithium diisopropylamide). Compound 2 (1 g, 0.0042 mol) was dissolved in 10 ml tetrahydrofuran, and the obtained LDA solution was further added thereto. 3 hours later, after the reaction was completed, it was quenched with 5 ml saturated NH₄Cl. The pH value of the reaction solution was adjusted to 3 with concentrated HCl, and an organic phase was separated by adding 10 ml ethyl acetate, washed with water (10 ml×3) and dried. After that, the organic phase was distilled under reduced pressure to remove the solvent. The residue was crystallized with methanol to obtain the subject product.

### Example 3

AlCl₃ (1.7 g, 0.013 mol) was dissolved in 10 ml dichloromethane in a 50 ml 3-neck flask at normal temperature, then the solution was cooled to 0° C. And diisopropylamine (2.8 g, 0.028 mol) was dissolved in 10 ml dichloromethane, then the resulting solution was added into the solution of AlCl₃ in dichloromethane, and the temperature of the mixed solution was kept below 20° C. Then it was warmed to room temperature naturally. Compound 2 (2 g, 0.0084 mol) was dissolved in 10 ml dichloromethane, and the solution was added into the reaction flask to react overnight. After the reaction was completed, it was quenched with 50 ml water, then the reaction system was filtrated and separated. And the organic phase was dried with anhydrous sodium sulfate and distilled under reduced pressure to remove the solvent, and then the residue was crystallized with methanol to obtain the subject product.

### Example 4

AlCl₃ (108 g, 0.81 mol) was dissolved in 250 ml chlorobenzene in a 1 L 3-neck flask at normal temperature, then the solution was cooled to 0° C. And diisopropylamine (152.9 g, 1.50 mol) was added into the solution of AlCl₃ in dichloromethane, and the temperature was kept below 20° C. Then it was warmed to room temperature naturally. Compound 2 (40 g, 0.17 mol) was dissolved in 150 ml chlorobenzene, and the solution was added into the reaction flask. The reaction lasted for at least 16 hours. After the reaction was completed, it was quenched with 1 L water, then the reaction system was filtrated and separated. Then the organic phase was dried with anhydrous sodium sulfate and distilled under reduced pressure to remove the solvent, the residue was crystallized with methanol to obtain the subject product. Yield (37 g, 64.9%), purity (97.3%).

### 2. Synthesis of Compound 1 (Racemic Tolterodine Free Base)

### Example 5

LiAlH₄ (0.27 g, 0.0072 mol) was added to 20 ml tetrahydrofuran in a reaction flask under the protection of nitrogen, then a solution of compound 3 (0.8 g, 0.0024 mol) dissolved in 10 ml tetrahydrofuran was added thereto, and the reaction mixture was heated to reflux (65° C.) and kept overnight. After the reaction was completed, it was quenched with 10 ml ethyl acetate and 2 ml water. Then the reaction system was filtrated, the organic phase was washed by water (10 ml×3), dried and distilled under reduced pressure to remove the solvent. The product was yellow oil, i.e., racemic tolterodine.

¹H-NMR (400 MHz, CDCl₃): δ 7.38 (m, 5H); 6.89 (m, 2H); 6.65 (s, 1H, J=7.6 Hz); 4.57 (dd, 1H); 3.28 (m, 2H); 2.78 (m, 1H); 3.42 (m, 2H); 2.16 (s, 3H); 2.10 (m, 1H); 1.18 (dd, 12H).

### Example 6

NaBH₄ (0.033 g, 0.0009 mol) was added to 5 ml tetrahydrofuran in a reaction flask under the protection of nitrogen, and compound 3 (0.1 g, 0.0003 mol) was added into the flask with stirring. Then the reaction mixture was cooled to 0° C. and H₂SO₄ (0.044 g, 0.00045 mol) was added therein. Half an hour later, it was heated to reflux (65° C.) and kept overnight. After the reaction was completed, it was quenched with 5 ml water, then an organic phase was separated by adding 5 ml ethyl acetate, and the aqueous phase was further extracted by 5 ml ethyl acetate. The organic phases were combined, adjusted with 2 mol/L HCl till the pH value thereof was 1 and heated to 70° C. 2 hours later, it was cooled to room temperature and the pH value thereof was adjusted to 12 with 20% NaOH. After that, the organic phase was separated again, washed by water (5 ml×3), dried and distilled under reduced pressure to remove the solvent. The product was yellow oil, i.e., racemic tolterodine.

### Example 7

NaBH₄ (19.3 g, 0.51 mol) was added to 80 ml tetrahydrofuran in a reaction flask under the protection of nitrogen, and compound 3 (19.5 g, 0.057 mol) was added into the flask with stirring, then the reaction mixture was cooled to 0° C. and a solution of AlCl₃ in tetrahydrofuran (23 g, 0.17 mol, dissolved in 80 ml THF) was added therein. Half an hour later, it was heated to reflux (65° C.). After the reaction was completed, it was quenched with 10% HCl and the pH value of the reaction system was adjusted to 1. After the reaction system was heated to 70° C., it was cooled to room temperature 2 hours later. Then the reaction system was adjusted by 20% NaOH till the pH value thereof was 12, and then extracted by dichloromethane twice (150 ml×2). The organic phase was washed by water (50 ml×3), dried and distilled under reduced pressure to remove the solvent to obtain the product as a yellow oil, i.e., racemic tolterodine. Yield (14.9 g, 80%). The crude product could be purified by hydrochlorination, wherein the purity was 98% or more.

### Example 8

Compound 3 (0.1 g, 0.0003 mol) was dissolved in 5 ml tetrahydrofuran in a reaction flask and the solution was cooled to 0° C. 3 ml borane-dimethylsulfide complex (0.003 mol) was dissolved in 5 ml tetrahydrofuran and the solution was added to the reaction flask, and the temperature was kept below 5° C. After the reaction was completed, it was quenched with 5 ml water, and the pH value of the reaction system was adjusted to 3 by HCl. Then an organic phase was separated by adding 5 ml ethyl acetate, and the aqueous phase was further extracted by 5 ml ethyl acetate. The organic phases were combined and the pH value thereof was adjusted to 1 with HCl. Then the organic phase was heated to 70° C., and 2 hours later, it was cooled to room temperature. The pH value thereof was adjusted to 12 with 20% NaOH and an organic phase was separated again. The organic phase was washed by water (5 ml×3), dried and distilled under reduced pressure to remove the solvent. The product was a yellow oil, i.e., racemic tolterodine.

### Example 9

To a 3-neck flask was added 5′ ml toluene, after charging nitrogen for half an hour, 1 g (0.003 mol) red-Al solution was added therein and the reaction mixture was cooled to 0° C. Compound 3 (0.1 g, 0.0003 mol) was dissolved in 5 ml toluene, and the solution was added to the reaction flask dropwise, after that, the temperature was increased to reflux temperature and the reaction lasted for 10 hours. Then the reactant was cooled to room temperature, and the pH value thereof was adjusted to 12 by adding 20% NaOH slowly, and then 10 ml dichloromethane was added thereto. The reaction mixture was stirred for half an hour, filtrated and washed by 10 ml water for 3 times, then dried and distilled under reduced pressure to remove the solvent. The product was a yellow oil.

### 3. Synthesis of Compound 4 (L-(+) Tartrate of Tolterodine)

### Example 10

Racemic tolterodine free base 1 (13 g, 0.04 mol) was dissolved in 80 ml acetone, and the solution was heated to reflux at 58° C. L-(+) tartaric acid (6.64 g, 0.44 mol) was dissolved in 40 ml methanol under reflux and the solution was added to the solution of tolterodine in acetone under the state of reflux, the reflux was kept for 1 hour. The reaction system was then cooled to 0° C. and kept for 1 hour. After that, it was filtrated to obtain the product as a white solid. The optical rotation [α]_{D}²³ was 28.5° C., and that reported in literature (U.S. Pat. No. 5,922,914) was 27.4° C.

The fundamental principle, main features and advantages of the present invention were described and illustrated as above. A person skilled in the art should understand that the present invention is not limited to the above examples. The examples and the embodiments described in the description are to be construed as merely illustration for principles of the present invention. It will be apparent to an ordinary person in the art that various changes and modifications which are falling into the scope claimed by the present invention may be made without departing from the spirit and scope of the invention. The protection scope of the present invention is defined by the accompanying claims.

1. A process for preparing tolterodine and the L-tartrate thereof, characterized in that the process includes the following steps:
A) performing ammonolysis reaction between diisopropylamine and 3,4-dihydro-6-methyl-4-phenyl-2H-benzopyran-2-one after diisopropylamine was activated by an activator, to give N,N-diisopropyl-3-phenyl-3-(2-hydroxy-5-methylphenyl)propionamide; or performing ammonolysis reaction between diisopropylamine and 3,4-dihydro-6-methyl-4-phenyl-2H-benzopyran-2-one after they were activated by Lewis acid, to give N,N-diisopropyl-3-phenyl-3-(2-hydroxy-5-methylphenyl)propionamide, and
the corresponding reaction equation is shown as follows: 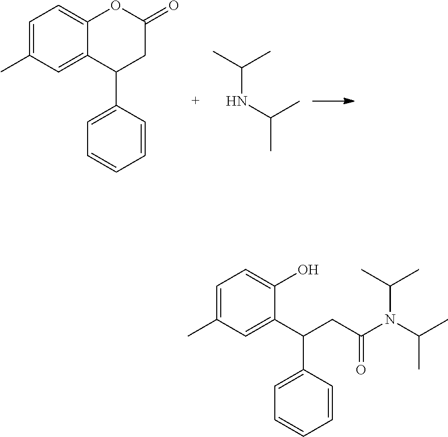
B) reducing N,N-diisopropyl-3-phenyl-3-(2-hydroxy-5-methylphenyl)propionamide obtained from the step A) by a reductant to obtain racemic tolterodine free base, and the corresponding reaction equation is shown as follows: 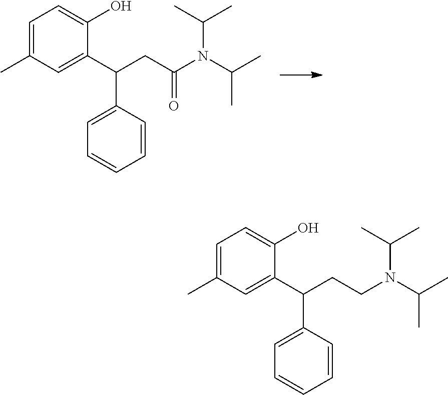
C) resolving racemic tolterodine free base by L-(+) tartaric acid to give L-(+) tartrate of tolterodine, and the corresponding reaction equation is shown as follows:

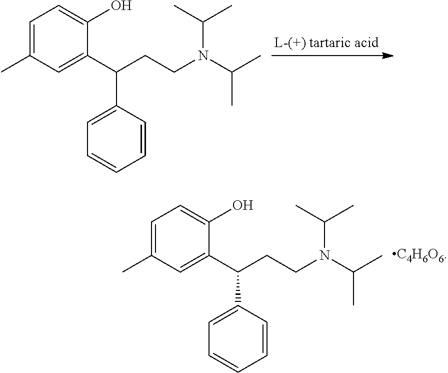

2. The process according to claim 1, characterized in that the activator used in the step A) is an alkali metal reagent selected from lithium aluminium hydride, butyllithium and Grignard reagent; and the molar ratio of the activator to diisopropylamine is 1:2 to 1:1.

3. The process according to claim 1, characterized in that the said Lewis acid used in the step A) is anyone of AlCl₃, ZnCl₂, BF₃, TiCl₄, Ti(O-i-Pr)₄, SnCl₄ and the combination thereof, and the amount of the Lewis acid is that the molar ratio of the Lewis acid to diisopropylamine is 1:1.5 to 1:5.

4. The process according to claim 1, characterized in that, in the step A), the solvent used in the ammonolysis reaction is tetrahydrofuran, acetonitrile, dichloromethane, toluene, xylene, chloroform, dichloroethane or chlorobenzene; the proper reaction temperature is from −80° C. to 130° C.; the molar ratio of 3,4-dihydro-6-methyl-4-phenyl-2H-benzopyran-2-one to diisopropylamine is 1:1 to 1:30.

5. The process according to claim 4, characterized in that, in the step A), the proper reaction temperature is from 10° C. to 70° C.; the molar ratio of 3,4-dihydro-6-methyl-4-phenyl-2H-benzopyran-2-one to diisopropylamine is 1:2 to 1:10.

6. The process according to claim 1, characterized in that, in the step A),
at the end of the step A), in the case of using an activator to activate diisopropylamine, the reaction is quenched with a quenching reagent, then the reaction liquid is acidified until the pH value thereof reaches 2 to 4, followed by extraction with organic solvent, then the extracted organic phase is concentrated under reduced pressure, after that, a crystallizing solvent is added thereto for crystallization to obtain N,N-diisopropyl-3-phenyl-3-(2-hydroxy-5-methylphenyl)propionamide;
in the case of using Lewis acid to activate diisopropylamine and 3,4-dihydro-6-methyl-4-phenyl-2H-benzopyran-2-one, after the reaction completes, it is quenched with water, then an organic phase is separated and concentrated under reduced pressure, after that, a crystallizing solvent is added thereto for crystallization to obtain N,N-diisopropyl-3-phenyl-3-(2-hydroxy-5-methylphenyl)propionamide;
wherein, the quenching reagent is anyone selected from water, aqueous solution of ammonia chloride, hydrochloric acid, sulfuric acid, formic acid, acetic acid, propionic acid, butyric acid, oxalic acid, citric acid and the combinations thereof; the acidification employs anyone of hydrochloric acid, sulfuric acid, formic acid, acetic acid, propionic acid, butyric acid, oxalic acid, citric acid and the combinations thereof; the organic solvent used for extraction is ethyl acetate, toluene, dichloromethane or methyl tert-butyl ether; the crystallizing solvent is anyone of ethyl acetate/petroleum ether, methanol, methanol/water, methyl tert-butyl ether/petroleum ether, methyl tert-butyl ether/heptane, dichloromethane/petroleum ether and the combinations thereof.

7. The process according to claim 1, characterized in that the reductant used in the step B) is anyone of metal hydride and borohydride, or the combinations of said hydrides and Lewis acid.

8. The process according to claim 1, characterized in that the reductant used in step B) is anyone of Red-Al, diisobutyl aluminium hydride and BH₃, or BH₃.(CH₃)₂S or BH₃.(CH₂)₄O as a complex of BH₃.

9. The process according to claim 1, characterized in that the reductant used in the step B) is NaBH₄, KBH₄, or any combination between either of them and a Lewis acid selected from sulfuric acid, AlCl₃, ZnCl₂, BH₃—(CH₂)₄O and I₂.

10. The process according to claim 1, characterized in that, in the step B), after the reduction reaction completes, it is quenched with water as the quenching reagent; then the reaction liquid is extracted with ethyl acetate or dichloromethane, and the resulting organic phase is concentrated under reduced pressure to obtain racemic tolterodine free base.
